(19) United States
(12) Patent Application Publication
(10) Pub. No.: US 2026/0198968 A1
(43) Pub. Date: Jul. 16, 2026

(54) SYSTEM AND METHOD FOR PARAURETHRAL SUPPORT RESTORATION TO TREAT STRESS INCONTINENCE

(71) Applicant: Simplified Surgical Solutions, LLC, Pompano Beach, FL (US)

(72) Inventor: Guillermo Davila, Fort Lauderdale, FL (US)

(21) Appl. No.: 19/397,131

(22) Filed: Nov. 21, 2025

Related U.S. Application Data
(63) Continuation-in-part of application No. 17/804,510, filed on May 27, 2022, now abandoned.
Application No. 17/804,510 is a continuation of application No. 17/435,932, filed on Jun. 5, 2020, now Pat. No. 11,376,041.
(60) Provisional application No. 62/857,918, filed on Jun. 6, 2019.

(51) Int. Cl. A61B 17/34; A61B 17/00; A61B 17/04; A61B 17/06; A61B 90/00

(52) U.S. Cl. CPC A61B 17/3468; A61B 17/0482; A61B 17/0483; A61B 90/08; A61B 2017/00805; A61B 2017/06176; A61B 2090/0811

(57) ABSTRACT

Disclosed are a system and method to implement a novel treatment to restore bilateral longitudinal parallel paraurethral support, the system comprising a trocar, and one or more barbed suture-type devices having a soft tissue anchor, the components used cooperatively to implant the one or more barbed suture-type devices in the patient to provide sufficient paraurethral support to restore continence in the patient.

### CLAIM OF PRIORITY

This application is a continuation-in-part of U.S. patent application Ser. No. 17/804,510 filed on May 27, 2022, which is a continuation of U.S. patent application Ser. No. 17/435,932 filed on Sep. 2, 2021, now U.S. Pat. No. 11,376,041, which is a National Stage Entry of International Application No. PCT/US2020/36266 filed on Jun. 5, 2020, which claims the benefit of U.S. provisional patent application Ser. No. 62/857,918 filed on Jun. 6, 2019, the contents of all of which are incorporated herein by reference.

### FIELD OF INVENTION

The present invention is directed to treatments and devices for incontinence. More specifically, disclosed are devices and methods for treatment of female stress incontinence through restoration of paraurethral support.

### BACKGROUND ART

In normal women, the urethra is supported by an interaction of the pelvic floor musculature, endopelvic fascia and bilateral attachments to the backside of the pubic symphysis and anterior pelvis. Until recently, the focus for treatment of incontinence caused by impaired urethral support has been on providing mid-urethral support by re-creating pubo-urethral ligaments which provide support at the level of the pelvic floor/levator musculature.

Prior to that, the most commonly used anti-incontinence procedure attached the proximal urethra and bladder neck to the ipsilateral Cooper's ligament with multiple sutures—and was termed a retropubic urethropexy or Burch colosuspension.

Specifically, current methods focus on mid-urethral sling procedures which kink the mid-urethra when under strain. Success rates in restoring continence are at most 90%, suggesting that there is more to the mechanism of urethral support responsible for continence.

Recently, greater degrees of understating of urethral support have focused on maintenance of urethral length providing support to the mechanism of action of retropubic urethropexy. In nulliparous women, longitudinal bilateral attachments to the posterior aspect of the pubic symphysis are identifiable as paraurethral indentations extending from the distal urethra to the bladder neck at the superior edge of the pubic bone.

This paraurethral support maintains urethral length and kinking at the bladder neck during strain maneuvers such as coughing. With the vaginal delivery process and repetitive life-long increases in intra-abdominal pressure, the paraurethral support mechanism becomes attenuated resulting in impaired urethral support, excess urethral mobility, and the development of stress urinary incontinence.

### SUMMARY OF INVENTION

The object of the present invention is to restore bilateral longitudinal parallel paraurethral support in order to restore continence in women with stress incontinence due to loss of paraurethral support.

The present invention provides a system, and a method of use to implement a novel treatment to restore bilateral longitudinal parallel paraurethral support, the system comprising at least one rigid trocar, and one or more flexible barbed suture-type devices with a soft tissue anchor. These components are used cooperatively to implant the one or more barbed suture-type devices in the patient bilaterally parallel to the urethra and attached to suprapubic support structures including fascia and ligaments (i.e,, Cooper's ligament). Once properly implanted the barbed suture-type devices provide sufficient paraurethral support to restore continence in the patient.

In one aspect of the system is a system for restoration of paraurethral support in a female patient comprising: a substantially rigid trocar comprising a lumen portion having an inside diameter, a proximal end, a distal end with a distal opening into said lumen, a handle extending from the proximal end of the lumen portion, the trocar adapted for insertion of the distal end of the lumen portion laterally to the female patient's urethra and advancement of the distal end of the lumen portion to the top of the female patient's pubic bone; and a semi-rigid, bendable barbed suture-type device comprising a central elongated suture thread having a proximal end and a distal end, a plurality of barbs extending from the suture thread, and a length, the distal end terminating in a soft tissue anchor, the barbed suture-type device adapted for insertion within the trocar, the length of the suture thread being greater than the length of the trocar lumen portion.

In some embodiments, the lumen portion of the trocar is curved to match the anatomical shape of the female patient. In other embodiments, at least some of the barbs extend in the direction of the proximal end of the suture thread. In further embodiments, at least some of the barbs extend in the direction of the distal end of the suture thread. In yet another embodiment, at least some of the barbs extend in the direction of the proximal end of the suture thread and at least some of the barbs extend in the direction of the distal end of the suture thread. In one further embodiment, the handle of the trocar further comprises an anchor release mechanism adapted to release the barbed suture-type device. In one other embodiment, the soft tissue anchor of the barbed suture-type device is larger than the distal opening of the trocar.

In another aspect described is a method for treating incontinence in a female patient comprising the steps of: loading a substantially rigid trocar with a semi-rigid bendable barbed suture-type device, the trocar comprising a lumen portion having a proximal end, a distal end with a distal opening into said lumen, and a handle extending from the proximal end of the lumen portion, the barbed suture-type device comprising a central elongated suture thread having a proximal end and a distal end terminating in a soft tissue anchor, and a plurality of barbs extending from the suture thread, the barbed suture thread adapted for insertion into the distal opening of the trocar; inserting the lumen portion of trocar loaded with the barbed suture-type device laterally to the female patient's urethra; advancing of the lumen portion of the trocar loaded with the barbed suture-type device substantially parallel to the female patient's urethra until the distal end of the trocar's lumen portion reaches the top of the female patient's pubic bone; engaging and securing the soft tissue anchor of the distal end of the barbed suture-type device to the female patient's tissue; and releasing the barbed suture-type device from the trocar and removing the trocar while the barbed suture-type device remains in place in the tissue of the female patient.

In at least some embodiments of the method, at least some of the barbs extend in the direction of the proximal end of the suture thread. In other embodiments, at least some of the barbs extend in the direction of the distal end of the suture thread. In further embodiments, at least some of the barbs extend in the direction of the proximal end of the suture thread and at least some of the barbs extend in the direction of the distal end of the suture thread. In at least one embodiment, the step of releasing the barbed suture-type device from the trocar further comprises pressing an anchor release mechanism on the handle of the trocar.

### BRIEF DESCRIPTION OF DRAWINGS

FIG. 1 illustrates a trocar component in accordance with one embodiment of the present invention.

FIG. 2A illustrates a trocar component having received a barbed suture-Type device in accordance with another embodiment of the present invention.

FIG. 2B illustrates an enlarged view of a distal end of the trocar component having received a barbed suture-type device of FIG. 2A in accordance with one embodiment of the present invention.

FIG. 3 illustrates an optional introducer component in accordance with one embodiment of the present invention.

FIG. 4 illustrates a barbed suture-type device component in accordance with one embodiment of the present invention.

FIG. 5A illustrates a barbed suture-type device component in accordance with another embodiment of the present invention.

FIG. 5B illustrates an enlarged view showing a plurality of barbs of a barbed suture-type device in accordance with one embodiment of the present invention.

FIG. 6 illustrates a cross sectional view of the female urethra showing general target placements for the barbed suture-type device components in accordance with one embodiment of the present invention. The circles indicate two different options for the general target placement of the barbed suture-type device.

FIGS. 7A and 7B illustrate, respectively, frontal and sagittal views of the bladder with barbed suture-type device components of one embodiment in place bilaterally in accordance with one embodiment of the present invention.

FIG. 8 illustrates a sagittal view of the top of the urethra with barbed suture-type device components of one embodiment in place bilaterally in accordance with one embodiment of the present invention.

### DESCRIPTION OF EMBODIMENTS

### System Features and Construction:

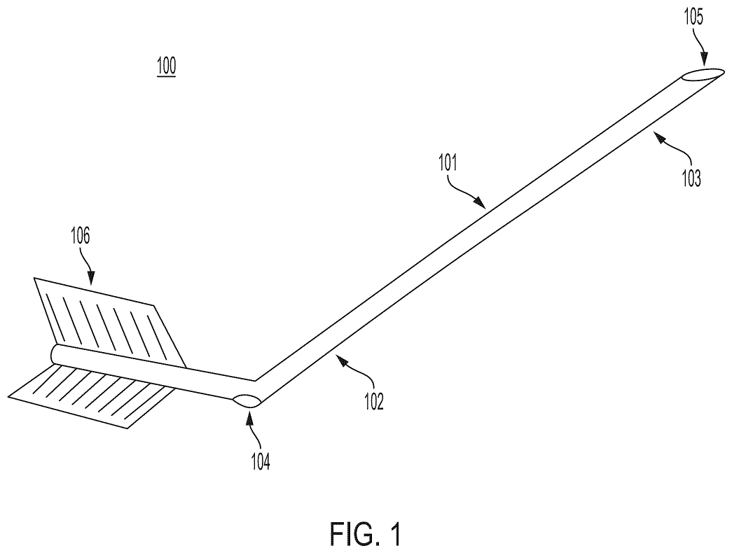

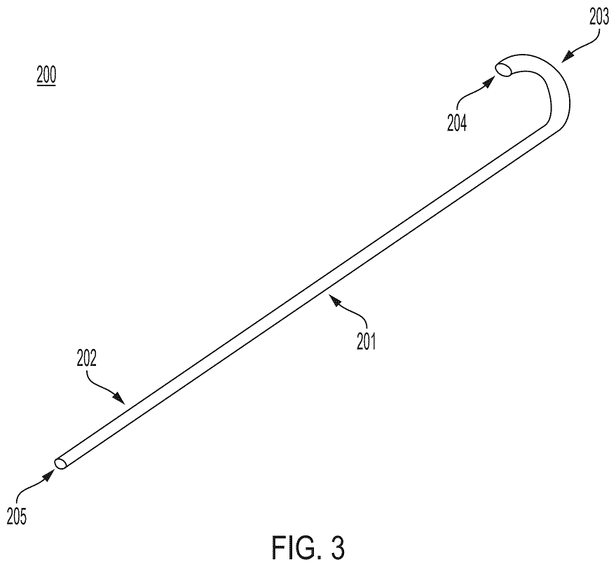

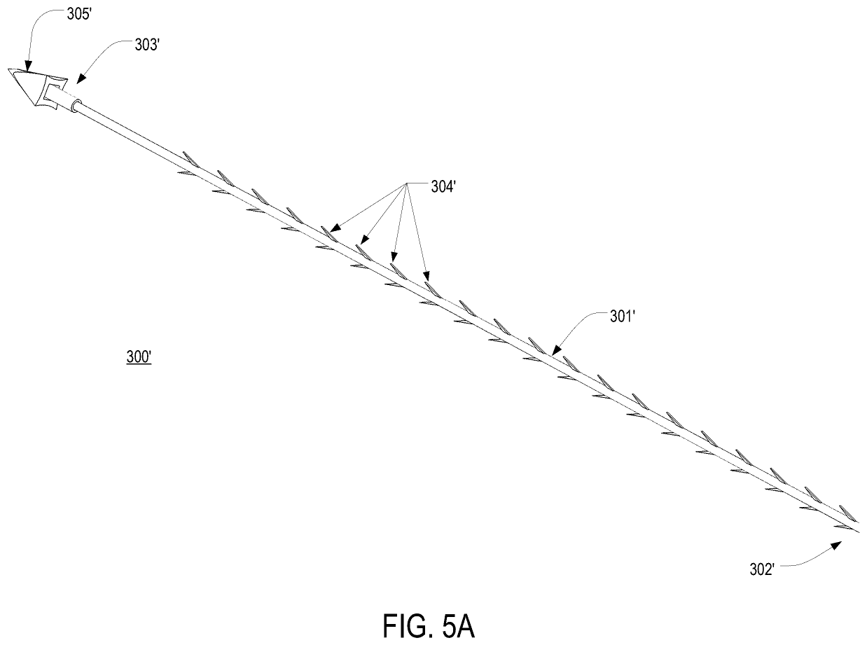

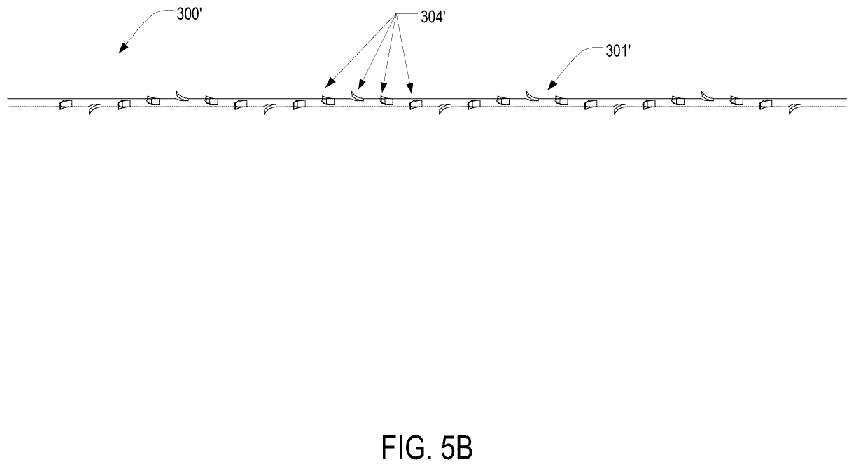

Referring to FIGS. 1, 2A, 2B, 4, 5A and 5B, the component devices of the system of the present invention comprise at least one trocar (100,100′), and at least one barbed suture-type device (300, 300′). Referring to FIG. 5A, in at least some embodiments, the at least one barbed suture-type device comprises an attached soft tissue anchor (305′). Referring to FIG. 3, in other embodiments, the system may optionally further comprise at least one introducer (200). Because in almost all situations the barbed suture-type device (300,300′) will be implanted bilaterally to the urethra, at least two barbed suture-type devices (300,300′) will be required in most cases. However, it is not beyond the scope of the present invention to utilize a single barbed suture-type device (300,300′) applied unilaterally. Moreover, where additional support is required, multiple barbed suture-type devices (300,300′) may be implanted on either, or both, sides of the urethra. Therefore, it is also possible for the system to comprise more than two barbed suture-type devices (300,300′).

The purpose of the trocar (100,100′) is to aid in the implanting of the barbed suture-type device (300,300′) along the urethral length to restore physiologic paraurethral support. In embodiments having an introducer (200), the introducer (200) may optionally also aid in the implanting of the barbed suture-type device (300) along the urethral length.

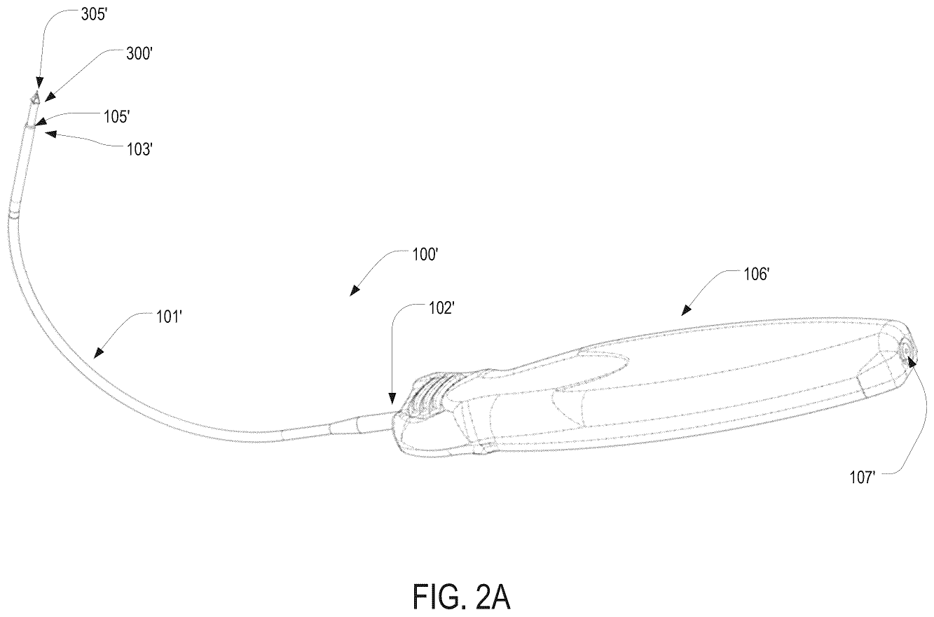

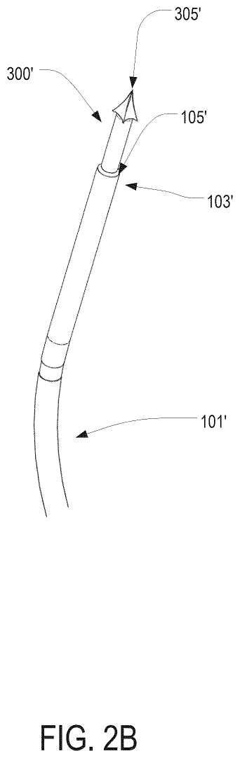

Referring to FIGS. 1, 2A and 2B, the trocar (100,100′) comprises a lumen portion (101,101′) having proximal (102,102′) and distal (103,103′) ends as well as a distal opening (105,105′). Referring to FIG. 1, in some embodiments, the trocar (100) may optionally also have a proximal opening (104). Referring again to FIGS. 1 and 2A, the lumen portion (101,101′) may be straight or curved to accommodate anatomical variations. A handle (106,106′) attached near the proximal end (102,102′) is adapted to permit manipulation of the trocar (100,100′) during use. In embodiments of the trocar (100′) adapted to directly receive the barbed suture-type device (300′), the handle (106′) may further comprise an anchor release mechanism (107′) (see FIG. 2A) adapted to release the barbed suture-type device (300′). The anchor release mechanism (107′) may optionally be an end or top push mechanism. The tip of the distal end (103,103′) of the lumen portion (101,101′) may optionally be sharpened to ease introduction, and, referring to FIGS. 2A and 2B, the tip of the distal end (103,103′) may also allow for placement of the soft tissue anchor (305′) in embodiments in which the barbed suture-type device (300′) comprises a soft tissue anchor (305′). Referring further to FIGS. 2A and 2B, the bore of the lumen portion (101,101′) is large enough to accommodate the outside diameter of the barbed suture-type device (300,300′) with the barbs (304,304′) collapsed and is adapted to be inserted on either side of the urethral meatus. In embodiments having an introducer (200), the bore of the lumen portion (101,101′) may be large enough to accommodate the outside diameter of the introducer (200). The length of the lumen portion (101,101′) is sufficient to advance from the meatus to the top of the pubic bone. Trocar (100,100′) is substantially rigid and may be made up from a number of different surgically suited rigid materials including metals, polycarbonates, and the like.

Referring to FIG. 3, optional introducer (200) comprises a substantially elongated hollow cylindrical body (201) having proximal and distal ends (202,203), as well as distal and proximal openings (204,205) disposed therein. The body is of sufficient length to traverse the entire length of the lumen portion (101) of the trocar (100). The outer diameter of the body (201) must be small enough to fit within the lumen portion (101). The inner diameter of the body (201) must be large enough to permit at least one barbed suture-type device (300) fit within it. The distal end (203) of the introducer (200) may optionally be curved to facilitate placement of the barbed suture-type device (300) during the procedure and to prevent bladder wall perforation. The introducer (200) may optionally also include indicia (not shown) on the proximal end (202) to indicate the orientation of the curved distal end (203). The construction of the introducer (200) is substantially flexible but resilient. The optional curved tip can be straightened for introduction into, and advancement through the lumen portion (101) of the trocar (100) but will immediately spring back to its curved shape when it emerges from the distal opening (105) of the trocar (100). The optional introducer (200) may be made up from a number of different surgically suited flexible materials including nitinol, polycarbonates, metals, and the like.

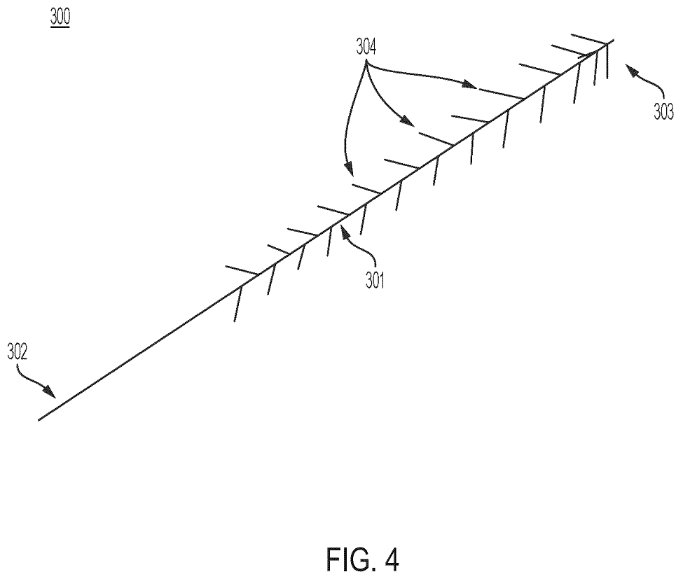

Referring to FIGS. 4 and 5A, barbed suture-type device (300,300′) comprises a central elongated suture-type thread (301,301′), having a proximal end (302,302′) and a distal end (303,303′), optionally with a soft tissue anchor (305′) attached at the distal end (303′) (FIG. 5A), and a plurality of short barbs (304,304′) extending from the thread (301,301′). In some embodiments the barbs (304′) extend from the thread (301′) along the proximal portion of the barbed suture-type device (300′) (see FIG. 5A). The barbs (304,304′) may extend in the direction of the proximal end (302,302′) or in the direction of the distal end (303,303′). Barbs (304,304′) extending in the direction of the proximal end (302,302′) will provide, once implanted in the paraurethral tissue, resistance to movement in the proximal direction. Barbs (304,304′) extending in the direction of the distal end (303,303′) will provide, once implanted in the paraurethral tissue, resistance to movement in the distal direction. Referring to FIG. 5B, a plurality of barbs (304′) are shown as being optionally distributed radially along the suture-type thread (301′). The amount of resistance to movement, and thus the level of support, provided by the barbed suture-type device (300,300′), can be adjusted by varying the flexibility of the barb material, or by changing the density of the barbs (304,304′) within the thread. Moreover, barb (304,304′) flexibility, density, direction, and placement may vary along the length of the thread (301,301′) to provide various levels of resistance along the length of the device.

The barbed suture-type device (300,300′) comprises a semi-rigid but bendable material, such as polypropylene, and the like. The barbed suture-type device (300,300′) is of a size adapted to be loaded into either the trocar (100′) (see FIGS. 2A and 2B), or optionally into the introducer (200) and advanced through the trocar (100) (e.g. size A #1 to #3 sutures) to be positioned along the length of the urethra from the top of the pubic bone, with attachment of the abdominal wall fascia, to the external urethral meatus. Distal end (303,303′) of the barbed suture-type device (300) may optionally comprise a needle tip, soft tissue anchor (305′), or dart (not shown) adapted to penetrate through the ipsilateral abdominal wall fascia (i.e., Cooper's ligament).

### Method of Use:

The procedure of the present invention may be performed in the doctor's office or in the surgical suite. A diagnosis of stress urinary incontinence is made. Urethral mobility is assessed. The following method is described in connection with the barbed suture-type device (300′) having a soft tissue anchor (305′) (see FIG. 5A) that is adapted to be received directly by the trocar (100′) (see FIGS. 2A and 2B).

The patient is placed in stirrups and a Foley catheter is placed in the bladder. A rigid catheter guide can optionally be placed into the foley catheter lumen in order to clearly identify the location of the urethra during the surgical procedure.

Local anesthetic (e.g. 1% lidocaine) is injected along the length of the urethra to the bladder neck and suprapubic lower abdominal wall bilaterally.

The barbed suture-type device (300′) is loaded onto the trocar (100′) by inserting the distal end (303′) of the barbed suture-type device (300′) into the distal opening (105′) of the trocar (100′). The barbed suture-type device (300′) is pushed through the trocar (100′) until the soft tissue anchor (305′) of the barbed suture-type device (300′) sits on the end of the trocar (100′).

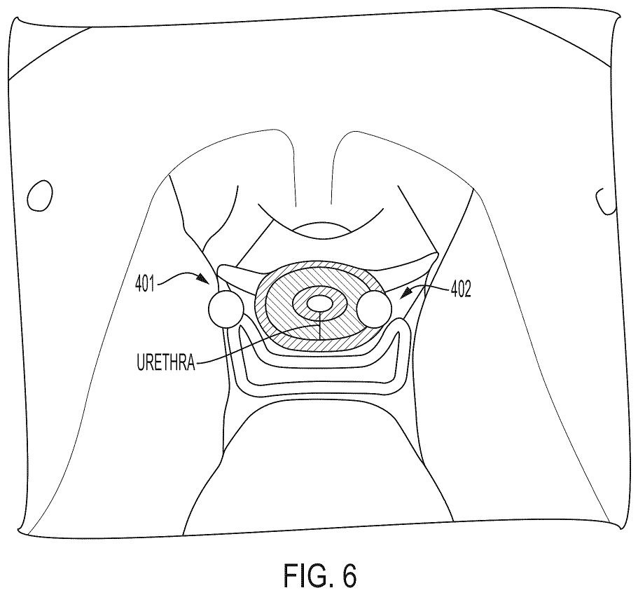

The trocar (100), pre-loaded with the barbed suture-type device (300′) having a soft tissue anchor (305′), is inserted a few millimeters lateral the urethral meatus (see FIG. 6, locations 401 and 402) and advanced along the urethra to the top of the pubic bone and until it indents the abdominal fascia and is palpable through the abdominal wall skin. The trocar can be directed towards the medial portion of the ipsilateral Cooper's ligament. The soft tissue anchor (305′) of the barbed suture-type device (300′) perforates the fascia along the top of the pubic bone (i.e., Cooper's ligament). This is performed bilaterally. Referring to FIG. 6, the left circle indicating general target placement (401) is illustrated as generally under the vaginal skin lateral to the urethra, and the right circle indicating general target placement (402) is illustrated as generally in the muscularis of the urethra itself. It should be noted that both general target placements (401,402) are acceptable placements, as well as other placements such that one of ordinary skill in the art would understand. If the method is performed bilaterally, then each barbed suture-type device should be placed in the same general target placement on either corresponding side. For example, each barbed suture-type device may be placed generally under the vaginal skin lateral to the urethra (e.g., 401) on the left and right sides, or each barbed suture-type device may be placed generally in the muscularis of the urethra itself (e.g., 402) on the left and right sides.

Cystoscopy is performed with a 70 scope to confirm bladder wall integrity. If the bladder was perforated, the trocar (100′) is repositioned, and cystoscopy repeated.

The loaded trocar (100′) is advanced until it is positioned at the desired position, possibly through Cooper's ligament and even full thickness through the abdominal wall skin and held in place. The trocar (100′) is then removed being careful to leave the barbed suture-type device (300′) in place along the insertion tract.

Any excess suture material is cut at the level of the external urethral meatus and abdominal wall skin if that is perforated. If the skin does not seal itself, tissue glue can be applied.

### Additional Observations:

The barb (304,304′) direction on the barbed suture-type device (300,300′) can vary depending on support needs. For example, facing proximally at one end and distally at the other.

More than one barbed suture-type device (300,300′) per side of the urethra may be required to enhance support.

The distal and/or proximal end (302,302′,303,303′) of barbed suture-type device (300,300′) may require a higher density of polypropylene and type of barbs in order to enhance support.

The barbed suture-type device (300,300′) may be advanced over top of pubic bone or into Cooper's ligament (using an optional curved tip introducer (200)) or simply towards abdominal wall (using an optional straight tip introducer (not shown)). A soft tissue anchor (305′) may be used at the distal end (303,303′) of barbed suture-type device (300,300′) to enhance support. The soft tissue anchor (305′) may have variable configurations and constructions of polypropylene or other biocompatible materials.

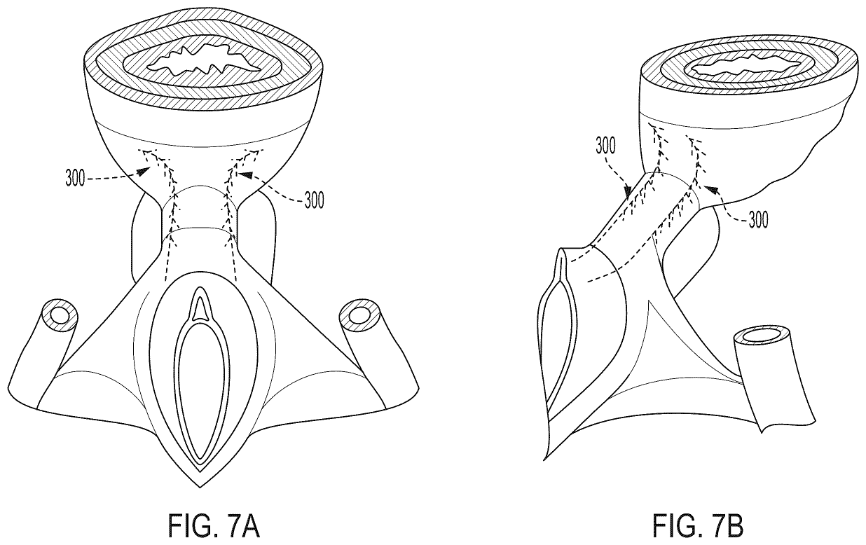

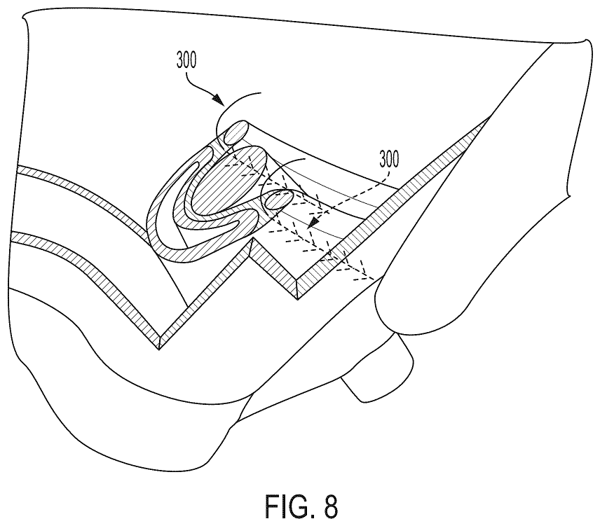

Shown in FIGS. 7A and 7B respectively, are frontal and sagittal views of the bladder with barbed suture-type devices (300) of FIG. 4 shown in place bilaterally after placement using the above-described method. FIG. 8 illustrates a sagittal view of the top of the urethra with barbed suture-type devices (300) of FIG. 4 shown in place bilaterally in accordance with the present invention. Although FIGS. 7A, 7B and 8 are depicted with the barbed suture-type device (300) of FIG. 4, it is to be understood that the barbed suture-type device (300′) of FIGS. 5A and 5B may also be used and placed accordingly.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the described embodiments, features, benefits, methods, and examples, but by all embodiments and methods within the scope and spirit of the invention as described and claimed.

Although described above in connection with treating stress incontinence, these descriptions are not intended to be limiting, as other systems and methods can be made in accordance with the description herein, but of different size or scale, for use in other surgical procedures, as desired. As such, although the system and method are illustrated and described herein, various modifications and structural changes may be made therein without departing from the spirit of the system or method and within the scope and range of equivalents of the claims. Further, any steps described herein may be carried out in any desired order (and any additional steps may be added as desired and/or any steps may be deleted as desired).

Any reference in this specification to “one embodiment,” “an embodiment,” an “example embodiment,” etc., means that a particular feature, structure, or characteristic described in connection with the implementation is included in at least one embodiment of the system or method. The appearances of such phrases in various places in the specification are not necessarily referring to the same embodiment. In addition, any elements or limitations of any system or method or embodiment thereof disclosed herein can be combined with any and/or all other elements or limitations (individually or in any combination) or any system or method or embodiment thereof disclosed herein, and all such combinations are contemplated with the scope of the system and method without limitation thereto.

As used herein, the terms “substantial” and “substantially” refer to the complete or nearly complete extent or degrees of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context, which one of ordinary skill in the art would be familiar with. However, generally speaking the nearness of completion will be so as to have the same overall result as if absolute or total completion were obtained.

The use of term “substantial” or “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

Although described above in connection with particular configurations, these descriptions are not intended to be limiting as various modifications may be made therein without departing from the spirit of the system or method and within the scope and range of equivalents of the described embodiments. Encompassed embodiments of the present system and method can be used in all surgical applications calling for insertion of a suture-type device.

While a number of embodiments of the present system and method have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from many different suitable materials.

In addition, various modifications and additions can be made to the exemplary embodiments discussed without departing from the scope of the present system or method. For example, while the embodiments described above refer to particular features, the scope of this system and method also includes embodiments having different combinations of features and embodiments that do not include all of the described features. Accordingly, the scope of the present system and method are intended to embrace all such alternatives, modifications, and variations as fall within the scope of the claims, together with all equivalents thereof.

I claim:

1. A system for restoration of paraurethral support in a female patient comprising:
a substantially rigid trocar comprising a lumen portion having an inside diameter, a proximal end, a distal end with a distal opening into said lumen, a handle extending from the proximal end of the lumen portion, the trocar adapted for insertion of the distal end of the lumen portion laterally to the female patient's urethra and advancement of the distal end of the lumen portion to the top of the female patient's pubic bone; and
a semi-rigid, bendable barbed suture-type device comprising a central elongated suture thread having a proximal end and a distal end, a plurality of barbs extending from the suture thread, and a length, the distal end terminating in a soft tissue anchor, the barbed suture-type device adapted for insertion within the trocar, the length of the suture thread being greater than the length of the trocar lumen portion.

2. The system of claim 1 wherein the lumen portion of the trocar is curved to match the anatomical shape of the female patient.

3. The system of claim 1 wherein at least some of the barbs extend in a direction of the proximal end of the suture thread.

4. The system of claim 1 wherein at least some of the barbs extend in a direction of the distal end of the suture thread.

5. The system of claim 1 wherein at least some of the barbs extend in a direction of the proximal end of the suture thread and at least some of the barbs extend in a direction of the distal end of the suture thread.

6. The system of claim 1 wherein the handle of the trocar further comprises an anchor release mechanism adapted to release the barbed suture-type device.

7. The system of claim 1 wherein the soft tissue anchor of the barbed suture-type device is larger than the distal opening of the trocar.

8. A method for treating incontinence in a female patient comprising the steps of:
loading a substantially rigid trocar with a semi-rigid bendable barbed suture-type device, the trocar comprising a lumen portion having a proximal end, a distal end with a distal opening into said lumen, and a handle extending from the proximal end of the lumen portion, the barbed suture-type device comprising a central elongated suture thread having a proximal end and a distal end terminating in a soft tissue anchor, and a plurality of barbs extending from the suture thread, the barbed suture thread adapted for insertion into the distal opening of the trocar;
inserting the lumen portion of trocar loaded with the barbed suture-type device laterally to the female patient's urethra;
advancing of the lumen portion of the trocar loaded with the barbed suture-type device substantially parallel to the female patient's urethra until the distal end of the trocar's lumen portion reaches the top of the female patient's pubic bone;
engaging and securing the soft tissue anchor of the distal end of the barbed suture-type device to the female patient's tissue; and
releasing the barbed suture-type device from the trocar and removing the trocar while the barbed suture-type device remains in place in the tissue of the female patient.

9. The method of claim 8, wherein at least some of the barbs extend in a direction of the proximal end of the suture thread.

10. The method of claim 8, wherein at least some of the barbs extend in a direction of the distal end of the suture thread.

11. The method of claim 8, wherein at least some of the barbs extend in a direction of the proximal end of the suture thread and at least some of the barbs extend in a direction of the distal end of the suture thread.

12. The method of claim 8, wherein the step of releasing the barbed suture-type device from the trocar further comprises pressing an anchor release mechanism on the handle of the trocar.
